(19) United States
(12) Patent Application Publication
(10) Pub. No.: US 2020/0330797 A1
(43) Pub. Date: Oct. 22, 2020

(54) REAL TIME AND NON-INVASIVE IN VIVO DOSIMETRY AND TISSUE MONITORING USING ELECTRICAL IMPEDANCE TOMOGRAPHY FOR RADIATION THERAPY

(71) Applicants: Virginia Commonwealth University, Richmond, VA (US); Norfolk State University, Norfolk, VA (US)

(72) Inventors: Taeho Kim, Glen Allen, VA (US); Hargsoon Yoon, Yorktown, VA (US)

(21) Appl. No.: 16/534,024

(22) Filed: Aug. 7, 2019

Related U.S. Application Data
(60) Provisional application No. 62/716,040, filed on Aug. 8, 2018.

(51) Int. Cl. A61N 5/10; A61B 5/053; A61F 7/00; A61B 5/00

(52) U.S. Cl. CPC A61N 5/1067; A61B 5/0536; A61F 2007/0002; A61B 5/4064; A61F 7/00

(57) ABSTRACT

A method for radiation dosimetry includes measuring an impedance of a tumor and a tissue surrounding the tumor in a patient using electrical impedance tomography, radiating the tumor in the patient with an emitted dosage and with or without thermal treatment, measuring an impedance change attributable directly or indirectly to radiation in the tumor and/or the surrounding tissue using electrical impedance tomography and continuing, adjusting, or stopping radiation of the tumor based on the measure of impedance change.

### CROSS-REFERENCE TO RELATED APPLICATIONS

This application claims priority to U.S. Provisional Application 62/716,040 filed Aug. 8, 2018. The complete contents thereof are herein incorporated by reference.

### FIELD OF THE INVENTION

The invention is generally related to methods and systems for targeted radiotherapy using non-invasive, real time, in vivo dosimetry.

### BACKGROUND OF THE INVENTION

Exposure of biomolecules, biological cells and tissues to radiation in therapeutic treatment can cause ionizing effects and damage to the targeted area in the body. The nature and severity of this change depends on the delivered radiation dose as the function of radiation beam energy and fluence. Radiation can ionize molecules in tissues and also change the conditions of the environment or medium (e g. extracellular space geometry, temperature).

Due to high demand for patient-specific radiotherapy with high radiation doses to tumors and minimum toxicity to healthy organs, complicated/targeted radiotherapy treatments have been developed and clinically used as a standard of care. In addition to accurate radiotherapy delivery, precise evaluation of radiation dose to tumors and healthy organs and monitoring of treated tissues is critical to assure the quality of radiotherapy delivery. In vivo dosimetry directly monitors the radiation dose delivered to a patient during radiation therapy. It allows comparison of prescribed and delivered doses and thus provides a level of radiotherapy quality assurance.

In-vivo dosimetry in radiotherapy relates to the measurement of the radiation dose received by the patient during treatment, as opposed to ex vivo or in vitro dose measurements made either before or after the treatment using a phantom to represent the patient. Current in vivo dosimetry technology capable of measuring the total dose during a treatment session include diodes, metal-oxide semiconductor field effect transistors (MOSFETs), plastic scintillation detectors (PSDs), and electronic portal imaging devices (EPIDs).

In conventional radiotherapy, dosimeters are placed on the patient's skin, and the dose to a point of interest inside the patient is inferred from surface measurements. The dose at a point within the patient is inferred from the reading on the surface and compared with a calculation which can include numerous serious errors from the radiotherapy system and incorrect and non-repeatable patient set up. Combined in-vivo measurements at both entrance and exit points is highly susceptible to errors in patient thickness and body shape changes, and the dose calculation algorithm or data in the planning system.

As a surrogate of in-vivo dosimetry, such dosimeters can provide only indirect information on the radiation dose to tumors unless the dosimeters are invasively implanted into a patient before the radiation delivery. Therefore, a true non-invasive in-vivo dosimeter is not available as a standard of care.

### SUMMARY

Embodiments of the disclosure provide for in-vivo dosimetry through a tomography technique with a non-invasive approach. The dosimetry described herein can improve the precision of radiotherapy delivery in real time and provide a tool of assessment leading to adaptive radiotherapy.

An aspect of the present disclosure provides a method for radiation dosimetry comprising measuring and imaging an impedance of a tumor and a tissue surrounding the tumor in a patient using electrical impedance tomography, radiating the tumor in the patient with an emitted dosage, measuring and imaging an impedance change attributable directly or indirectly to radiation in the tumor and/or the surrounding tissue using electrical impedance tomography and continuing, adjusting, or stopping radiation of the tumor based on the measure of impedance change.

In some embodiments, the continuing or adjusting includes redirecting a path of the radiation and/or adjusting the emitted dosage when an impedance change is detected in the surrounding tissue. In further embodiments, the impedance change is an increase in conductivity of at least 0.3% between a baseline and sequential measurement which is due to an ionization effect from radiation therapy. If the effects from radiation-induced extracelullar volume and temperature increases are included, the amount of conductivity increase will be added to the value further. In some embodiments, adjusting includes adjusting the emitted dosage when an impedance change outside of a predetermined acceptable range is detected in the tumor. In some embodiments, the steps of radiating the tumor and measuring the impedance change due to radiation may be performed simultaneously. In some embodiments, the steps of radiating the tumor and measuring the impedance change due to radiation are repeated for a plurality of cycles. In some embodiments, at least six electrodes are used for the electrical impedance tomography. In some embodiments, the tumor and surrounding tissue are in a portion of the patient's brain, but not limited.

Another aspect of the present disclosure provides a computer-executable storage medium whose contents cause a computing system to perform a method for radiation dosimetry as disclosed herein.

Another aspect of the present disclosure provides a system for radiation dosimetry comprising a plurality of electrodes fixable to a patient for electrical impedance tomography, a radiation delivery source configured to provide radiation therapy to the patient, one or more computer-executable storage media containing computer-executable instructions, and one or more processors that, upon execution of the computer-executable instructions, cause the system to perform a method for radiation dosimetry as disclosed herein. In some embodiments, the system further comprises an output device for providing results of the electrical impedance tomography measurements.

### BRIEF DESCRIPTION OF THE DRAWINGS

FIG. 1. Schematic of real-time in-vivo dosimetry (radiation dose monitoring and measurement) using electrical impedance tomography in radiotherapy according to some embodiments of the disclosure.

FIG. 2. An example linear accelerator system according to some embodiments of the disclosure.

FIG. 3. Schematic of an irradiation pulse train with 3 μs irradiation time for 100 Hz repetition rate.

FIG. 4. Schematic of an exemplary radiation therapy procedure according to some embodiments of the disclosure.

FIG. 5. Schematic description of direct ionization of molecules and formation of radicals upon irradiation.

FIG. 6. Electrochemical impedance model. C_{dl}: double layer capacitance, Z_{w}: Warburg resistance, R_{ct}: charge transfer resistance, Rₘ: membrane resistance, Cₘ: membrane, capacitance, Rₑₓ: extracellular resistance.

### DETAILED DESCRIPTION

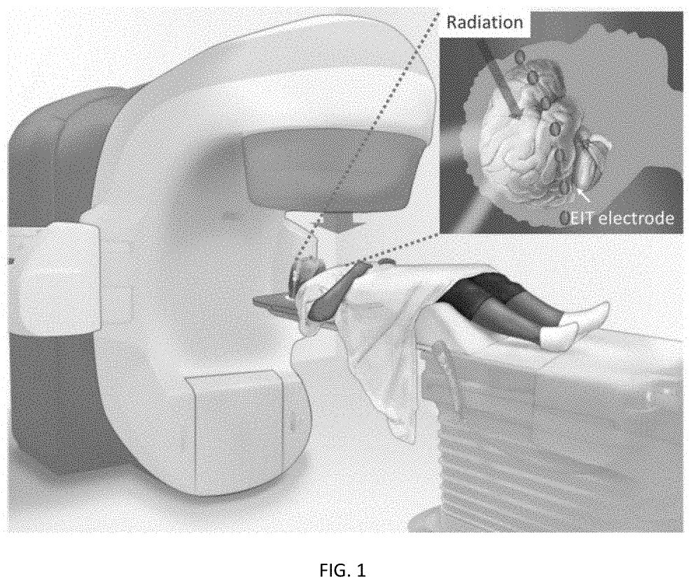

Embodiments of the disclosure provide methods for measuring and monitoring an internal radiation dose and tissue responses in real-time during radiation therapy using electrical impedance tomography (FIG. 1). Through this application, precise radiation therapy can be achieved by a real-time feedback mechanism. In addition, internal dosimetry based radiotherapy assessments leads to effective adaptive radiotherapy. The tissue being targeted for radiation therapy may move within the body because of breathing, organ filling or emptying, or other internal movement. Embodiments of the disclosure allow for accurate monitoring of the radiation beam and dosage to insure accurate delivery of radiation to the target and, if needed, only a minimum margin around the target.

Radiation therapy is the use of high-energy radiation to damage cancer cells' DNA and destroy their ability to divide and grow. The radiation delivery source may be an ionizing radiation device, known as a linear accelerator (LINAC), for external beam radiation therapy but could be any radiation therapy delivery device including radioactive sources placed inside the patient on a temporary or permanent basis as for internal radiation therapy. Embodiments of the disclosure are compatible with all types of radiation therapy.

Exemplary radiation therapy delivery devices include such devices manufactured by Varian Medical Systems, Inc. of Palo Alto, Calif.; Siemens Medical Systems, Inc. of Iselin, N.J.; Electa Instruments, Inc. of Iselin, N.J.; or Mitsubishi Denki Kabushik Kaisha of Japan. Such devices may be used to deliver conventional single or multi-field radiation therapy, 3D conformal radiation therapy (3D CRT), inverse modulated radiation therapy (IMRT), stereotactic radiotherapy, or tomo therapy. This may be done in conjunction with a variety of treatment planning software systems. Prior to therapy, the location of the tumor may be determined by electrical impedance tomography or by other methods known in the art such as computed tomography (CT) and magnetic resonance imaging (MRI).

Ionizing radiation for external beam radiation therapy can be grouped into 2 major types: photon radiation (x-rays and gamma rays) and particle radiation (such as electrons, protons, neutrons, carbon ions, alpha particles, and beta particles).

For photon radiation, a high-energy photon beam is often utilized and is the same type of radiation that is used in x-ray machines. Photon beams of energy affect the cells along the beam path as the beam passes through the body to get to the cancer, passes through the cancer, and then exits the body.

Proton beams are a form of particle beam radiation. Positively charged protons release their energy only after traveling a certain distance and cause little damage to the tissues they pass through. This makes proton therapy very efficient to deliver more radiation damage to targetted cancer cells while minimizing damage to nearby normal tissues.

A neutron is a particle in many atoms that has no charge. Neutron beam radiation therapy is applied to some cancers of the head, neck, and prostate and certain tumors that other forms of radiation therapy are ineffective to treat.

Carbon ion radiation may also be referred to as heavy ion radiation because it uses atomic particles that are heavier than protons or neutrons. Carbon ion radiation can be effective because heavier ions can do more damage to the target cell than other types of radiation. As with protons, the beam of carbon ions can be adjusted to do the most damage to the cancer cells at the end of its path. In contrast, there is a higher entrance dose compared to proton radiotherapy so the effects on nearby normal tissue may be enhanced.

A medical linear accelerator (LINAC) is the device most commonly used for external beam radiation treatments. It delivers high-energy x-rays or electrons to the region of tumor. As a patient lies on a table, the linear accelerator moves around the patient to deliver radiation from several angles. The linear accelerator can be adjusted for a patient's particular situation so that it delivers the prescribed radiation dose precisely. Patients typically receive external beam radiation on an outpatient basis five days a week over a certain period of time. In most instances, treatments are usually spread out over several weeks to allow healthy cells to recover in between radiation therapy sessions. Each treatment session may last approximately 10 to 30 minutes. In some cases, a single treatment may be used to help relieve pain or other symptoms associated with more-advanced cancers. To provide precise treatment and minimize motion during the treatment, a patient might be positioned with molds to hold the patient's body in place. The patient will lie still and breathe normally during the treatment. For some patients with lung or breast cancer, the patient might be asked to hold their breath while the machine delivers the treatment.

The LINAC may be used to treat all body sites, using conventional techniques such as Intensity-Modulated Radiation Therapy (IMRT), Volumetric Modulated Arc Therapy (VMAT), Image Guided Radiation Therapy (IGRT), Stereotactic Radiosurgery (SRS) and Stereotactic Body Radio Therapy (SBRT). In similarity, particle therapies using protons and heavy ions may be used to treat various disease sites using 3D conformal or intensity-modulated techniques.

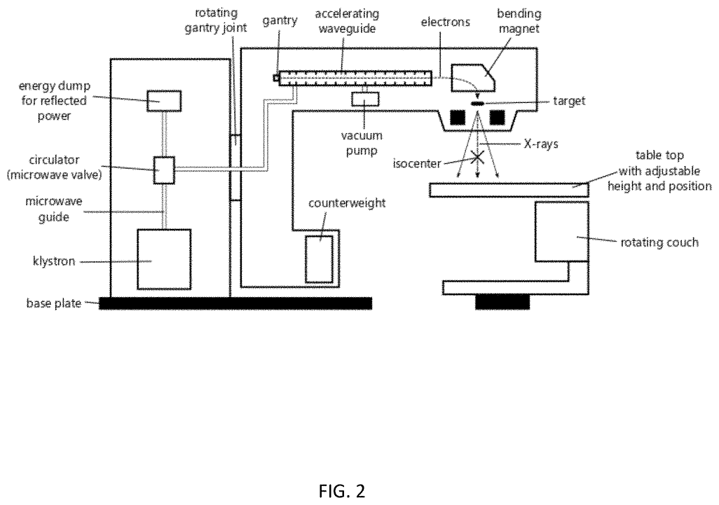

The linear accelerator accelerates to produce high-energy as shown, for example, in FIG. 2. The high energy beam is shaped as it exits the machine to conform to the shape of the patient's tumor and the customized beam is directed to the patient's tumor. The beam is usually shaped by a multileaf collimator that is incorporated into the head of the machine. The beam comes out of a part of the accelerator called a gantry, which can be rotated around the patient. Radiation can be delivered to the tumor from many angles by rotating the gantry and moving the treatment couch.

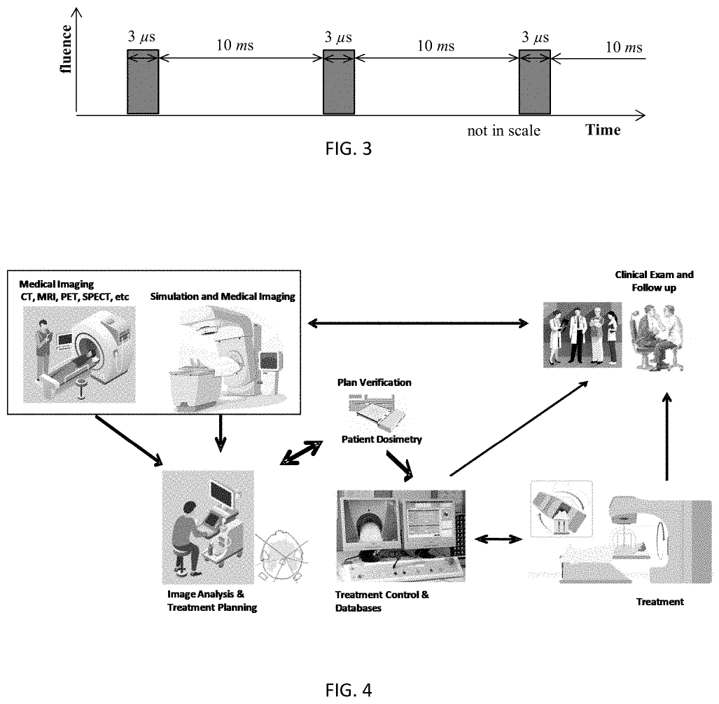

An example linear accelerator that is commercially available is the ELEKTA SYNERGY® LINAC system which delivers radiation with an energy range between 6 to 18 MV for photon and 4 to 18 MeV for electron beams, and irradiating treatment fields for X-rays until 40 cm×40 cm and electrons until 25 cm×25 cm, respectively. The pulse width is 3 μs and pulse repetition frequency (PRF), the number of pulses per second, is 6, 12, 25, 50, 100, 200 and 400 Hz. FIG. 3 illustrates a 3 μs irradiation time for 100 Hz repetition rate.

Preparation and planning for radiation therapy is focused on targeting the radiation dose to the cancer as precisely as possible to minimize side effects and avoid damaging normal cells. FIG. 4 provides a schematic of an exemplary radiation therapy procedure. Imaging tests are used to help determine the exact shape and location of the tumor and define its boundaries. Before external beam radiation therapy, patients may undergo computerized tomography (CT) scans and/or MRI to determine the area of body to be treated. With the images, a radiation therapy team decides what type of radiation and what dose patients will receive based on the type and stage of cancer, general health conditions, and the goals for treatment. The precise dose and focus of radiation beams used in the treatment is carefully planned to maximize the radiation to cancer cells and minimize the harm to surrounding healthy tissue.

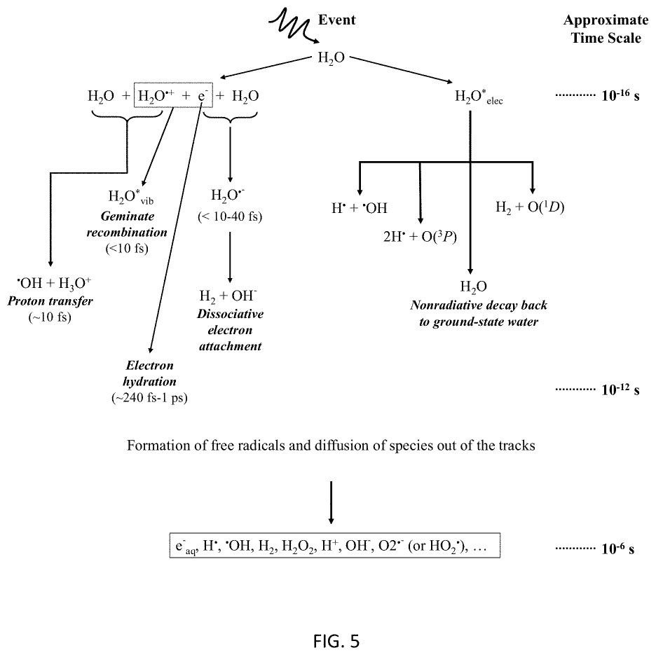

With reference to FIG. 5, exposure of biomolecules, biological cells and tissues to radiation in therapeutic treatment can cause ionizing effects and damage to the targeted area in the body. The biological effectiveness of radiation depends on the linear energy transfer (LET), fluence, total dose, number of fractions and radiosensitivity of the targeted cells or tissues. These cellular and medium changes under the radiation beam can be measured by the methods disclosed herein.

Ionization is the dissociation of molecules by cleavaging one or several chemical bonds resulting from exposure to high-energy beam flux. Radiation induced ionizations can act directly on the cellular molecules and cause damage (direct effect). Free radicals also form in water by two mechanisms: excitation or ionization at the individual molecular level and can attack other critical molecules (indirect effect)¹. Water is the major (˜80%) constituent of cells. The changes of charged ions and free radicals can be quantitatively measured by the methods disclosed herein.

For example, the amount of radiation used in radiation therapy is measured in grays (Gy), and varies depending on the type and stage of cancer being treated. For curative cases, the typical dose for a tumor ranges from 2 to 10 Gy per fraction. Many factors are considered by radiation oncologists when selecting a dose, including whether the patient is receiving chemotherapy, patient comorbidities, whether radiation therapy is being administered before or after surgery, and the degree of success of surgery. Delivery parameters of a prescribed dose are determined during treatment planning.

For simplicity of calculation, conductivity change induced by radiation is calculated as an example, since water is the major (˜80%) constituent of cells. In conventional proton therapy, the therapeutic proton beam provide the energy range of 200-250 MeV and the flux that is ≥10¹⁰ s⁻¹. Assuming a pulsed proton beam at 200 MeV and a pulse repetition rate of 25 Hz with the flux 5×10¹⁰/cm²·s, the degree of ionization, the number of ion pairs formed per unit volume can be calculated. First, the range of proton energy Eₚ, 200 MeV in air is calculated using a semi-empirical formula as,

$${R}_{air} = {( \frac{{E}_{p}}{9.3} )}^{1.8} = {( \frac{200}{9.3} )}^{1.8} = 250.4 m$$

for the radiation energy of a few MeV to 200 MeV². Their range in water (i.e., the penetration depth into water) is calculated using the Bragg-Kleeman rule,

$${R}_{water} = 3.2 \times 1 {0}^{- 4} \frac{\sqrt{{M}_{water}}}{{ρ}_{water}} {R}_{air} = 34.1 cm .$$

Water requires 14.53 eV to create an ion pair, the number of ion pairs (IP), produced by the protons with the flux 5×10¹⁰/cm²·s is approximately,

$$IP = \frac{200 \times 1 {0}^{6} eV / proton}{14.53 eV / i . p .} ( 5 \times 1 {0}^{10} \frac{protons}{{cm}^{2} sec} ) \frac{1}{34.1 cm} = 2.02 \times 1 {0}^{16} \frac{ion pairs}{{cm}^{3} s e c} .$$

When the pulse repetition rate is 25 Hz, the number of ion generated during each pulse is 8.07×10¹⁴/cm³.

Considering sodium (Na⁺) with a normal range of 10-15 mM in the intracellular space and 140 mM in the extracellular space and chloride (Cl⁻) with a range of around 99˜110 mM as the major electrolyte constituents in the human brain³, the average charge concentration adding other mobile charge carriers in human brain tissue can be assumed to be around 3.0×10¹⁷/cm³. From this calculation, tissue conductivity change by the ionization itself is a decrease of about 0.3%, which is in the range of detection by electrical impedance tomography. For example, the tissue conductivity change due to radiation and/or thermal treatment may be a decrease of at least about 0.3%, 0.4%, 0.5%, 0.6%, 0.7%, 0.8%, 0.9%, 1.0%, or more. Typical conductivity values measured by EIT are as follows: liver 0.28-0.33 S/m, gray matter 0.22-0.30 S/m and white matter 0.16-0.23 S/m in the brain⁴. When the conductivity is compared with healthy tissue in white matter, tumors show a 40-180% conductivity increases. This can be generalized to all tumor types in that tumors typically have a 40-180% conductivity increase as compared to healthy tissues. By assuming similar ion concentrations in the healthy tissue, a change of impedance, i.e. an increase of conductivity between a baseline and sequential measurement by ionization, can be measured by the methods disclosed herein.

In addition to the radiation induced ionization, radiolysis and oxidizing events by ionizing radiation also alter biological cells and environment through direct interactions of radiation with target macromolecules or via products of water radiolysis⁶. After multiple pathways of ionization, the various chemically reactive species diffuse and react with one another or with the environment. Finally, the cells respond to the environmental changes resulting from the products formed in the preceding stages. During this stage (˜10⁻³ seconds or longer, depending upon the medium), the biological cells and tissue responses are damaged by the oxidative stress. To cope with the radiation induced stress and the changes in environment depending on radiation dose, dose rate and quality, the biological system elicits transient responses at the molecular, cellular and tissue levels⁶.

Regarding the dose relationship with biological cell responses, radiolysis and oxidizing events cause extracellular space (ECS) volume changes by osmotic stress. For example, each brain cell is separated by the ECS, which contains interstitial fluid, the extracellular matrix, and other secreted molecules⁷. The ECS forms a complex 3D structure and occupies ˜20% of the brain. The ECS is not a static compartment, but changes over the course of physiological stresses⁸. The ECS serves as a reservoir of ions for electrical activity and the physical corridor for diffusion-mediated transport of ions and ionic current flow. Radiation changes the cell environment (extracellular space volume and geometry) and extracellular medium (e g. ionic concentration and diffusion constant)^{9,10}. These cellular and medium changes can be measured by the methods disclosed herein.

Another effect of radiation is heating by absorption of radiation energy. In some embodiments of ionizing radiation of low linear energy transfer (LET), the heating effect may be negligible. The low radiation energy is deposited and localized in a small spherical region. For low-LET radiation the local temperature rise may be too small and too brief to have any appreciable chemical (or physical) effect¹¹. With high-LET ionizing radiations (such as fission fragments, stripped nuclei, and α-particles), a large amount of energy is deposited per unit path length, resulting in cylindrical tracks (rather than spherical spurs). The equation for temperature rise in this case is written as¹¹,

$$Δ T ( γ , t ) = Δ {{T}_{max} ( 1 + \frac{γ t}{{a}^{2}} )}^{- 1} exp ( - \frac{{γ}^{2}}{{a}^{2} + 4 γ t} ) ,$$

where a is the initial size parameter for the track cylinder and ΔTₘₐₓ is the maximum initial temperature rise on its axis. With this effect, high LET radiation results in a high degree of local heating, and, even though the heat pulse survives only a short time, that time still is long enough to bring about the acceleration of reactions between the short-lived intermediates and the ambient substrate.

The temperature change caused by high LET radiation brings a decrease in impedance in the area, due to the prominent increase in ion mobility of the extracellular fluid. The nature and severity of this change depends on the delivered radiation dose. Heating effects by radiation can be measured by methods disclosed herein.

In addition to the monitoring of radiation-induced heating effects, EIT measurement is also useful for hyperthermic treatments of tumors in which the tumor is heated to a temperature of about 40-45° C. or more. Such treatments may be performed alone or in combination with radiation treatments. Ionic conductivity from charge transport mainly by diffusion can be obtained by an equation below¹².

$$Re [ σ ( ω ) ] ≅ {σ}_{d c} [ 1 - ( \frac{56}{45} ) \sqrt{\frac{2 D}{ω {L}^{2}}} ]$$

where σ_{dc}, D, ω and L respectively represent the dc conductivity, diffusion constant, angular velocity and distance of current flowing medium. Since the diffusion constant D is temperature sensitive, the changing ratio of diffusion constant by temperature change from T₁ to T₂ can be obtained by the equation below¹³.

$$α = \frac{{D}_{2}}{{D}_{1}} = exp ( 0.02 ( {T}_{2} - {T}_{1} ) )$$

where D₁ and D₂ are diffusion contants at the temperature T₁ and T₂ respectively. When we assume the temperature change from 36° C. to 43° C., diffusion constant increase (α=1.15) around 15% is expected. In turn, increase of conductivity Re[σ(ω)] will be around 7.3% using the equation above. This conductivity variation can be measured by methods disclosed herein.

Electrical impedance tomography (EIT) is a noninvasive imaging technology in which electrical conductivity, permittivity, and impedance of a part of the body or an organ may be inferred from surface electrode measurements^{14,15}. EIT technology can measure bioelectrical impedance changes and produce an image representing spatial tissue impedance distribution inside a body or organ. Electrodes surround the region, and an electrode pair injects a sub-sensory current while potentials are measured on the remaining electrodes. At low frequencies, electrical current flows through the extracellular space without penetrating the cell membrane and induces changing of electrical current flow depending on the sensing domain impedance change.

Using this principle, arrays of electrodes can be placed on the body, e.g. the scalp, to measure environmental, physiological, anatomical or morphological changes of cells or tissues in the sensing domain prior to radiation therapy (baseline). A temperature increase caused by radiation brings a decrease in impedance in the area, due to a prominent increase in ion mobility. The measured impedance change can capture the difference in the group of cells from distant electrodes. The measured results may be incorporated into signal processing, and images may be generated using image reconstruction algorithms. By arranging electrodes in certain configurations and multiplexing signal acquisition, the system and methods described herein can concurrently perform recordings of impedance in several important structures and imaging of multiple targets in the sensing domain, while radiation therapy is operated (sequential measurements).

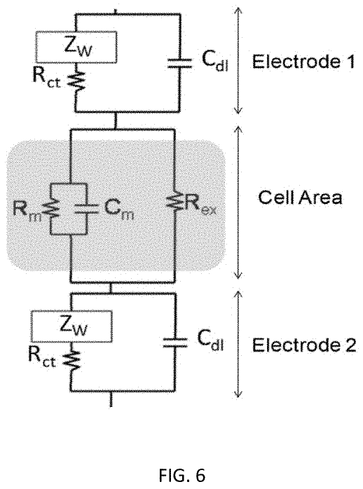

The electrical properties of living tissues may be modelled as a group of electronic components as shown in FIG. 6. As a simple example, the intra-cellular space, and the membrane are modelled as a resistor Rₘ and a capacitor Cₘ, respectively and the extracellular space is modelled as resistor Rₑₓ. Electrical properties of tissue under irradiation are continuously subject to change by many different sources including ionization of cell structures and changes of extracellular space (volume and geometry) and medium (e g. ionic concentration and molecular diffusion properties) due to oxidative stress and temperature changes. Embodiments of the disclosure are based on the measurement of those impedance changes.

For example, several changes within cells and extracellular medium such as changes in charged ions, free radicals and extracellular matrix will cause an impedance decrease. The generation of charged ions and free radicals will increase charge carriers that will also drop impedance values. Extracellular matrix changes will increase the volume and size of free charge carrier passages, which will also lower the impedance values. In addition, at high temperatures upon radiation, molecular diffusion and mobility of charged particles will enhance thus causing the impedance to decrease.

The amount of impedance change may be different for each patient's condition and tissue responsivity to radiation. Embodiments of the disclosure allow for visualizing the difference and changes in the target tissue and in the surrounding healthy tissues not irradiated. Using the methods disclosed herein, the path of the radiation beam and/or the degree/dosage of radiation may be adjusted in real time to avoid healthy tissue and/or excessive irradiation, based upon the differential images.

By this in-vivo EIT measurement, the dosimetric baseline is acquired prior to radiation delivery and the dosimetric information is determined from the difference between dosimetric baseline and sequential measurements in radiation delivery. The difference map may be presented in real time for in-vivo dosimetry and can be applied for adaptive radiation therapy. Recorded data in internal computer storage can be used for dosimetric evaluation after the radiotherapy.

Some embodiments of the disclosure provide a method for radiation dosimetry, comprising measuring an impedance of a tumor and a tissue surrounding the tumor in a patient using electrical impedance tomography, radiating the tumor in the patient with an emitted dosage, measuring an impedance change attributable directly or indirectly to radiation in the tumor and/or the surrounding tissue using electrical impedance tomography and continuing, adjusting, or stopping radiation of the tumor based on the measure of impedance change.

In some embodiments, the continuing or adjusting includes redirecting a path of the radiation and/or adjusting the emitted dosage when an impedance change is detected in the surrounding tissue to minimize collateral damage to the healthy tissue surrounding the tumor. Redirecting the path of the radiation may involve one or both of changing the position/angle of the radiation source and changing the position/angle of the patient. In some embodiments, adjusting includes adjusting the emitted dosage when an impedance change outside of a predetermined acceptable range (where the acceptable range is indicative of the desired dosage) is detected in the tumor. The emitted dosage should be adjusted to bring the impendance value within the acceptable range.

Radiating the tumor and measuring the radiation-induced impedance change may be performed simultaneously. In some embodiments, the steps of radiating the tumor, measuring the radiation-induced impedance change, and optionally redirecting a path of the radiation and/or adjusting the emitted dosage are repeated for a plurality of cycles over the course of a treatment session. The term “plurality”, as used herein, is defined as two or as more than two.

A system as described herein allows application of radiation therapy to a target at a selected dosage and intensity modulation with precise accuracy, while minimizing the margin needed around the target. For example, when Stereotactic Body Radio Therapy (SBRT) is applied, 5˜7 mm margin is included from clinical target volume (CTV). For the case of Intensity-Modulated Radiation Therapy (IMRT), 10 mm margin is included from CTV. Accuatrate dose evaluation and precise intensity modulation in adaptive radiotherapy will help to determine a proper margin and minimize the margin. In one embodiment, the impedance of the target and surrounding tissue is substantially continuously monitored during delivery of the radiation therapy. If the path of radiation moves away from the target beyond an acceptable range of displacement distances, the administration of radiation from the radiation delivery device can be interrupted, e.g. via a signal from a computer controller to the radiation delivery device. The position of the radiation beam can then be adjusted manually or automatically to refocus on the target, and radiation therapy can resume or continue. The range of movement is dependent upon many factors, such as the target type (e.g., brain, prostate, lung, liver), target size, target location, beam shape/size, and the radiation treatment plan.

Some embodiments provide a computer-executable storage medium whose contents cause a computing system comprising one or more processors to perform a method for radiation dosimetry as disclosed herein. A computer or processor may be operatively coupled to the EIT electrodes and the radiation delivery source and thus is configured to receive EIT readings, control the radiation dosage emitted from the radiation source, and control the movement of the radiation source and/or patient support/bed. The term “coupled”, as used herein, is defined as connected, although not necessarily directly, and not necessarily mechanically. All components of a system as described herein may be connected via wire and/or wirelessly.

Some embodiments provide a system for radiation dosimetry comprising a plurality of electrodes fixable to a patient for electrical impedance tomography, a radiation delivery source configured to provide radiation therapy to the patient, one or more computer-executable storage media containing computer-executable instructions, and one or more processors that, upon execution of the computer-executable instructions, cause the system to perform a method for radiation dosimetry as disclosed herein. The EIT readings may be displayed on an output device, e.g. a monitor, screen, speaker, or printer, for review by the attending physician/staff.

Components disclosed herein within a configured system or other embodiments disclosed herein may be implemented by a controller and data system of various circuitry. Such a controller and data system may be in the form of a desktop computer, a mobile device, laptop computer, a network server, a server computer, or may be implemented by any one of or a combination of general or special-purpose processors (digital signal processor (DSP)), firmware, software, and/or hardware circuitry to provide instrument control, data analysis, etc., for the example configurations disclosed herein.

Individual software modules, components, and routines may also often be utilized by disclosed systems in the form of a computer program, procedure, or process written in a suitable programming language, e.g. Java, C, C #, C++. In addition, the computer programs, procedures, or processes may be compiled into intermediate, object, or machine code and presented for execution as instructions and control functions so as to be implemented by a disclosed system herein, or any other configurations disclosed herein. Various implementations of the source, intermediate, and/or object code and associated data may also be stored in one or more computer executable storage media that include read-only memory, random-access memory, magnetic disk storage media, optical storage media, flash memory devices, and/or other suitable media. As used herein, the term “computer executable storage media” excludes propagated signals, per se and refers to media known and understood by those of ordinary skill in the art, which have encoded information provided in a form that can be read (i.e., scanned/sensed) by a machine/computer and interpreted by the machine's/computer's hardware and/or software.

EIT can generate cross-sectional images of impedance distribution of the body through a plurality of electrodes placed in a transverse plane over an area of the body. This is possible because the electrical conductivity of different body tissues varies widely (e.g., from 1.25 S·m⁻¹ for cerebrospinal fluid to 0.02 S·m⁻¹ for bone)¹⁶, so that an impedance distribution image may be formed.

To obtain a still image or video, a group of electrodes may be attached to a subject. The group consists of non-current-carrying and current-carrying electrodes. In an embodiment, the electrodes may be linked to a data acquisition unit that outputs the data, for example, to a PC or other computing device. In an embodiment, by applying a series of small currents to the current-carrying pairs of electrodes, a set of potential difference measurements may be made from non-current carrying pairs of electrodes. The electric currents applied to the body take the path of least impedance, where the currents' flow depends on the subject's conductivity distribution.

In an embodiment, an EIT system may be provided using, for example, 6 channels, 32 channels, 128 channels or more (or other numbers as desired). Such a system may use a single current source that may be switched electronically between any pair of electrodes. In an embodiment, the system may use parallel (simultaneous) measurement of the potential on the remaining (4, 30, 126, etc.) electrodes. For high sensitivity and resolution, the diameter of the electrodes can be from sub-millimeter. Flexible leads may be applied between the electrodes and electronics.

In an embodiment, the system may use a multi-frequency digitally synthesized injection current, with maximal frequency of, for example, 10 KHz to 1 GHz. The use of multiple scanning frequencies provides a conductivity spectrum for each tissue type, and thus provides further conductivity contrast to differentiate tissues from each other, making the entire EIT process more robust. In addition, higher scanning frequencies allow faster data acquisition and thus higher temporal resolution in object position tracking. For a high degree of versatility, the system may be set to record the raw potentials at a high sample rate, for example at four to ten times the maximal scanning frequency, depending on the steepness of the anti-aliasing filters. Such a setting may allow visualization of artifacts and noise, and thus may provide opportunities to reduce or remove them.

In an embodiment, several parameters of the system may be programmable, including the number and frequency of sine waves in the synthesized current injection waveform, the “dwell time” or switching speed between different injection pairs, the number and sequence of injection pairs used, and/or the current level.

In an embodiment, current level may be limited to the maximal allowed leakage current for medical devices appropriate for the frequency of current injection used. The gain of the measurement amplifiers may be set appropriately to capture the full dynamic range of skin surface potentials expected to be encountered. An anti-aliasing filter may be used to prevent high-frequency noise from being digitized along with the signal. In an exemplary embodiment, an analog-digital converter (e.g. a 24-bit converter) may be used and the sample rate of the converter (one per channel) may be set to match the roll-off of the filter. Phase shifts caused by the anti-aliasing filter may also be measured and factored into the analysis.

After digitization and multiplexing of the signal from each electrode, the data may be serialized and sent to an interface board in a controlling computer or processor, which may, in an embodiment, be placed at a considerable distance from the subject and treatment equipment.

In some embodiments, a floating AC current source is connected to skin electrode pairs via CMOS multiplexers. At any time, two electrodes may be driven with the current source and the remaining electrodes may be connected to low-noise preamplifiers to measure the voltage at each electrode. A digital controller (field programmable gate array (FPGA) or microprocessor) may sequence the multiplexers through all electrode combinations.

In some embodiments, a data acquisition and control system includes a multiplexer, amplifier, analog to digital converter, and a signal interface.

In some embodiments, a system includes a harnessing structure comprising electrodes to be mounted on the body, e.g. on the skull surface, for electrical currents. It is contemplated that a voltage supply may interfere with the trajectory of the radiation beam during radiation therapy. Device materials and designs for EIT can be carefully designed/manufactured to avoid any interference.

The methods disclosed herein may be used for primary tumors from a number of tumor sites, including, but not limited to, the brain, the lungs, the prostate, the liver, the breasts, melanoma, the pancreas, and the pelvis. In some embodiments, the location being treated is substantially stationary, e.g. a brain tumor. In other embodiments, the location is moving, e.g. the lungs and liver via respiratory action.

Embodiments of the disclosure are also useful for hyperthermic treatments of tumors in which the tumor is heated to a temperature of about 40-45° C. or more. Such treatments may be performed alone or in combination with radiation treatments.

While the system is described in connection with guided radiation therapy or hyperthermic treatment of a tumor, the system can be used for tracking and monitoring other targets within a body, such as for other therapeutic or diagnostic purposes.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which is further illustrate the invention, and are not intended, nor should they be interpreted to, limit the scope of the invention.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

1. Kyle G. Reeves and Yosuke Kanai, Electronic Excitation Dynamics in Liquid Water under Proton Irradiation, Scientific Reports, 7, pp. 40379, (2017). DOI: 10.1038/srep40379
2. N. Tsoulfanidis, Measurement and Detection of Radiation, Hemisphere Publishing Corp., pp. 126-128, (1983).
3. G. Madelin, R. Kline, R. Walvick, and R. R. Regatte, Ionic charge transport between blockages: Sodium cation conduction in freshly excised bulk brain tissue, Scientific Reports, 4, pp. 4763 (2014).
4. Hannah McCann, Giampaolo Pisano, Leandro Beltrachini, Variation in reported human head tissue electrical conductivity values, Brain Topography, pp. 1-34, (2019).
5. Huhndorf, M., Stehning, C., Rohr, A., Helle, M., Katscher, U., & Jansen, O., Systematic brain tumor conductivity study with optimized EPT sequence and reconstruction algorithm. Abstract #3626, (2013). Paper presented at the Proc. ISMRM in Salt Lake City, Utah, USA.
6. Edouard I. Azzam, Jean-Paul Jay-Gerin, and Debkumar Pain, Ionizing radiation-induced metabolic oxidative stress and prolonged cell injury, Cancer Lett., 327(0), pp. 48-60, (2012). doi:10.1016/j.canlet.2011.12.012.
7. Charles Nicholson, Diffusion and related transport mechanisms in brain Tissue, Rep. Prog. Phys. 64, pp. 815-884, (2001).
8. Sabina Hrabetova, Laurent Cognet, Dmitri A. Rusakov, and XU. Valentin Nagerl, Unveiling the Extracellular Space of the Brain: From Super-resolved Microstructure to In Vivo Function, The Journal of Neuroscience, 38(44), pp. 9355-9363, (2018).
9. Lia K. Jacobson, Maxwell B. Johnson, Raj D. Dedhia, SolmazNiknam-Bienia, Alex K. Wong, Impaired wound healing after radiation therapy: A systematic review of pathogenesis and treatment, JPRAS Open, 13, pp. 92-105, (2017).
10. SYKOVÁ, E., SVOBODA, J., SIMONOVÁ, Z., LEHMENKÜHLER, A. and LASSMANN, H. X-irradiation-induced changes in the diffusion parameters of the developing rat brain. Neuroscience 70, 597-612 (1996).
11. britannica.com/science/radiation/Heating-effects
12. David Emin, Massoud Akhtari, B. M. Ellingson, and G. W. Mathern, Ionic charge transport between blockages: Sodium cation conduction in freshly excised bulk brain tissue, AIP Advances 5, pp. 087133, (2015); https://doi.org/10.1063/1.4928652.
13. Charles Nicholson, Diffusion and related transport mechanisms in brain tissue, Rep. Prog. Phys. 64, pp. 815-884, (2001).
14. Holder, D. S., Feasibility of developing a method of imaging neuronal activity in the human brain: a theoretical review, Med. Biol. Eng Comput., 25, pp. 2-11, (1987).
15. Aristovich, K. Y., Packham, B. C., Koo, H., Santos, G. S. D., McEvoy, A., Holder, D. S., Imaging fast electrical activity in the brain with electrical impedance tomography, Neurolmage, 124, pp. 204-21, (2016).
16. C Gabriel, A Peyman and E H Grant, Electrical conductivity of tissue at frequencies below 1 MHz, Phys. Med. Biol. 54 (2009) 4863-4878.

We claim:

1. A method for radiation dosimetry, comprising:
measuring and/or imaging an impedance of a tumor and a tissue surrounding the tumor in a patient using electrical impedance tomography;
radiating the tumor in the patient with an emitted dosage;
measuring and/or imaging an impedance attributable directly or indirectly to radiation in the tumor and/or the surrounding tissue using electrical impedance tomography; and
continuing, adjusting, or stopping radiation of the tumor based on the measure of impedance change.

2. The method of claim 1, wherein continuing or adjusting includes redirecting a path of the radiation and/or adjusting the emitted dosage when an impedance change is detected in the tissue surrounding the tumor.

3. The method of claim 2, wherein the impedance change in the tissue surrounding the tumor is a decrease in impedance of at least 0.3%.

4. The method of claim 1, wherein adjusting includes adjusting the emitted dosage when an impedance change outside of a predetermined acceptable range is detected in the tumor.

5. The method of claim 1, wherein the steps of radiating the tumor and measuring the impedance change due to radiation are performed simultaneously.

6. The method of claim 1, wherein the steps of radiating the tumor and measuring the impedance change due to radiation are repeated for a plurality of cycles.

7. The method of claim 1, wherein at least six electrodes are used for the electrical impedance tomography.

8. The method of claim 1, wherein the tumor and the tissue surrounding the tumor are in a portion of the patient's brain.

9. The method of claim 1, further comprising administering a thermal treatment to the tumor, wherein the measured impedance change is due to either or both the radiation and the thermal treatment.

10. A computer-executable storage medium encoded with computer-executable instructions that cause a computing system to perform a method for radiation dosimetry, the method comprising:
measuring an impedance of a tumor and a tissue surrounding the tumor in a patient using electrical impedance tomography;
radiating the tumor in the patient with an emitted dosage;
measuring an impedance change attributable directly or indirectly to radiation in the tumor and/or the surrounding tissue using electrical impedance tomography; and
continuing, adjusting, or stopping radiation of the tumor based on the measure of impedance change.

11. The computer-executable storage medium of claim 10, wherein the continuing or adjusting includes redirecting a path of the radiation and/or adjusting the emitted dosage when an impedance change is detected in the tissue surrounding the tumor.

12. The computer-executable storage medium of claim 11, wherein the impedance change in the surrounding tissue is a decrease in impedance of at least 0.3%.

13. The computer-executable storage medium of claim 10, wherein the adjusting includes adjusting the emitted dosage when an impedance change outside of a predetermined acceptable range is detected in the tumor.

14. The computer-executable storage medium of claim 10, wherein the steps of radiating the tumor and measuring the impedance change due to radiation are performed simultaneously.

15. The computer-executable storage medium of claim 10, wherein the steps of radiating the tumor and measuring the impedance change due to radiation are repeated for a plurality of cycles.

16. The computer-executable storage medium of claim 10, wherein at least six electrodes are used for the electrical impedance tomography.

17. The computer-executable storage medium of claim 10, wherein the tumor and tissue surrounding the tumor are in a portion of the patient's brain.

18. The computer-executable storage medium of claim 10, wherein the method further comprises administering a thermal treatment to the tumor, wherein the measured impedance change is due to the radiation and the thermal treatment.

19. A system for radiation dosimetry, comprising a plurality of electrodes fixable to a patient for electrical impedance tomography;
a radiation delivery source configured to provide radiation therapy to the patient;
one or more computer-executable storage media containing computer-executable instructions; and
one or more processors that, upon execution of the computer-executable instructions, cause the system to perform steps comprising
measuring an impedance of a tumor and a tissue surrounding the tumor in the patient using electrical impedance tomography;
radiating the tumor in the patient with an emitted dosage;
measuring an impedance change attributable directly or indirectly to radiation in the tumor and/or the surrounding tissue using electrical impedance tomography; and
continuing, adjusting, or stopping radiation of the tumor based on the measure of impedance change.

20. The system of claim 19, further comprising an output device for providing results of the electrical impedance tomography measurements.
